(19) United States
(12) Patent Application Publication
(10) Pub. No.: US 2022/0000664 A1
(43) Pub. Date: Jan. 6, 2022

(54) IMPLANTATION DEVICE

(71) Applicant: Mayo Foundation for Medical Education and Research, Rochester, MN (US)

(72) Inventors: Jarel K. Gandhi, Modesto, CA (US); Alan D. Marmorstein, Rochester, MN (US); Jose S. Pulido, Rochester, MN (US); Justin T. Koepsel, Rochester, MN (US); Lucas A. Iezzi, Rochester, MN (US); Fukutaro Mano, Osaka (JP)

(21) Appl. No.: 17/428,703

(22) PCT Filed: Feb. 10, 2020
(86) PCT No.: PCT/US2020/017444

§ 371 Date: Aug. 5, 2021

Related U.S. Application Data
(60) Provisional application No. 62/803,108, filed on Feb. 8, 2019.

(51) Int. Cl. A61F 9/00; A61F 9/007; A61F 2/14

(52) U.S. Cl. CPC A61F 9/0017; A61F 2210/0076; A61F 2/14; A61F 9/007

(57) ABSTRACT

This document relates to an implantation device for cellular therapeutics and other therapeutic materials embedded on or in human fibrin or other polymer scaffolds. For example, this document relates to methods and devices for delivering iPSC-RPE monolayers grown on a fibrin scaffold into the sub-retinal space of the eye. The device includes a handle assembly including an actuator and a tip assembly at a distal end of the handle assembly. The tip assembly can house the implant, and the actuator is configured to eject the implant from the tip assembly.

### CROSS-REFERENCE TO RELATED APPLICATIONS

This application claims priority to U.S. Application Ser. No. 62/803,108, filed on Feb. 8, 2019. The disclosure of the prior application is considered part of the disclosure of this application, and is incorporated in its entirety into this application.

### BACKGROUND

### 1. Technical Field

This document relates to an implantation device for cellular therapeutics and other therapeutic materials embedded on or in human fibrin or other polymer scaffolds. For example, this document relates to methods and devices for delivering induced pluripotent stem cells-retinal pigment epithelium (iPSC-RPE) monolayers grown on a fibrin scaffold into the sub-retinal space of the eye.

### 2. Background Information

Macular degeneration represents a group of diseases that commonly result from retinal pigment epithelium (RPE) dysfunction. Genetic macular degenerations, including the bestrophinopathies, occur due to protein mutations involved in RPE function. While genetically-caused macular degenerations are rare, age-related macular degeneration (AMD) is the leading cause of blindness in the first world. It is estimated that AMD will account for 5 million cases in the US by 2050.

RPE replacement as a treatment for macular degeneration has been the subject of study for 30 years. Cadaveric and fetal sources of RPE for transplantation are however, limited. Pluripotent (PS) stem cells, both embryonic stem cells (ESCs) and induced pluripotent stem cells (iPSCs) have been shown in multiple reports to differentiate to an RPE lineage (Sonoda et al., Nat. Protoc., 4:662-673 (2009); Johnson et al., Ophthalmol. Vis. Sci., 56:4619 (2015); Brandl et al., NeuroMolecular Med., 16:551-564 (2014); Idelson et al., Cell Stem Cell., 5:396-408 (2009); Carr et al., Mol. Vis., 15:283-295 (2009)). Both ES-RPE and IPS-RPE have been shown to exhibit normal RPE function, including expression of phenotypic cell markers, phagocytosis of photoreceptor outer segments, and pigmentation (Singh et al., Ophthalmol. Vis. Sci., 54:6767-6778 (2013)).

### SUMMARY

This document relates to an implantation device for delivering engineered constructs. For example, this document relates to methods and devices for delivering fibrin-scaffolded iPSC-RPE monolayers into the sub-retinal space of the eye.

This document relates to RPE transplantation. While in vitro successes of RPE transplantation have been attained, many difficulties have risen in translation towards clinical application. The earliest trials attempted to deliver RPE single cell suspensions to the subretinal space in dry AMD patients (Peyman et al., Ophthalmic Surg., 22:102-108 (1991); and Schwartz et al., The Lancet., 379:713-720 (2012)). These studies showed safety efficacy, as no adverse reactions were reported (Schwartz et al., The Lancet., 379:713-720 (2012); Schwartz et al., The Lancet., 385:509-516 (2015); and Schwartz et al., Ophthalmol. Vis. Sci., 57: ORSFc1-9 (2016)). However, transplantation was characterized by low percentage of RPE attachment and survival. As expected, no major improvement was detected in visual acuity (Schwartz et al., The Lancet., 385:509-516 (2015)).

As an epithelium, cell-cell contact is involved in RPE survival and function. Subsequent trials have focused on the growth of RPE monolayers for transplantation. A recent study utilized collagen gel culture of RPE and use of collagenase to detach the monolayer as a single unit prior to transplantation (Kamao et al., Stem Cell Rep., 2:205-218 (2014); and Sun et al., Stem Cells, 33:1543-1553 (2015)). Animal studies transplanting the unsupported RPE monolayer with this model have shown an improvement in attached cell viability after transplantation. However, a concern presented was the inability to maintain a flat, wrinkle-free monolayer through the surgical procedure. As such, cell attachment is seen off target and with clumping phenotype. The first human trial with this strategy has been performed (Mandai et al., N Eng J Med., 376:1038-1046 (2017)), and the clinical trial is on-going.

Two methods of RPE transplantation have been examined. These are single cell suspensions and monolayers. Results from studies in humans in which a single cell suspension of RPE is injected into the subretinal space have demonstrated poor cell survival and little engraftment (Algvere P V, et al (1997) Graefes Arch Clin Exp Ophthalmol 235(3):149-158, Schwartz S D, Invest Ophthalmol Vis Sci 57 (5): ORSFc1-9 and Schwartz S D, et al. (2015) Lancet 385(9967):509-516). Studies using RPE monolayers have been shown to be superior with excellent rates of cell survival and even some demonstration of efficacy in early stage human clinical trials. Injection of free-floating monolayers of RPE however, leads to pilling, clumping, and possible inversion of the cells (Mandai M, et al. (2017) N Engl J Med 376(11):1038-1046). The solution used to overcome this is a scaffold which delivers the RPE as a flat monolayer. Different approaches to this have been applied. The first studies in humans have used permanent polymer scaffolds formed from paralyene or PET (da Cruz L, et al. (2018). Nat Biotechnol 36(4):328-337 and Kashani A H, et al. (2018) Sci Transl Med 10 (435)). Instruments used to deliver RPE on these materials have generally been of either a modified forceps design, or a modified metal or silicon cannula with either direct mechanical or hydraulic pressure to move the implant out of the device.

As surgical procedures advance and scaffolding material and scaffold dimensions change in response, new instruments are required. Thicker scaffolds cannot be folded, precluding the use of a forceps-like inserter instrument. In other instruments, the need for the surgeon to directly apply mechanical force via a thumb wheel or plunger could cause an unsafe loss of precision during placement of the implant. Finally, instruments using hydraulic force to deliver the implant may not be regulated with regard to fluid ejection and pressure (a significant safety issue) and are not feasible for use in delivering denser, heavier, implants in a safe manner. This document relates to an implantation device for RPE monolayers on scaffolds or other cellular or molecular materials embedded in or on a scaffold. For example, this document relates to methods and devices for delivering fibrin scaffolded iPSC-RPE monolayers into the sub-retinal space of the eye.

In one aspect, the disclosure is directed to a device for implanting an implant. The device includes a handle assembly including an actuator, and a tip assembly at a distal end of the handle assembly, the tip assembly configured to house the implant. The actuator is configured to eject the implant from the tip assembly.

In some embodiments, the actuator is a pneumatic actuator. In some embodiments, the pneumatic actuator is compatible with a vitrectomy unit. In some embodiments, the handle assembly receives the tip assembly. In some embodiments, the tip assembly is removably coupled to the distal end of the handle assembly. In some embodiments, the tip assembly is removably coupled to the distal end of the handle assembly via threads. In some embodiments, the tip assembly is disposable. In some embodiments, the tip assembly is preloaded with the implant. In some embodiments, the tip assembly includes a tip hub configured to couple to the handle assembly. In some embodiments, the tip hub includes a spring loaded pin. In some embodiments, the spring-loaded pin is in communication with the actuator, wherein the spring-loaded pin is configured to eject the implant from the tip assembly upon actuation of the spring-loaded pin. In some embodiments, the tip assembly further includes a tip housing and a tube extending between the tip hub and the tip housing. In some embodiments, the tube houses a guidewire extending between the tip hub and the tip housing. In some embodiments, the tip housing includes a plunger coupled to the guidewire, wherein the plunger is in communication with the actuator, wherein the plunger is configured to eject the implant from the tip housing upon actuation of the plunger.

In some embodiments, the tube houses a push rod extending between the hub and the tip housing. In some embodiments, the push rod has a generally flattened, oval-shaped cross section. In some embodiments, the tip housing is clear. In some embodiments, the tube is stainless steel. In some embodiments, the tube defines a radius of curvature. In some embodiments, the radius of curvature is about 3 mm to about 60 mm. In some embodiments, the tube further includes a seal, wherein the seal is configured to inhibit eye fluid from entering the tip hub. In some embodiments, the implant is a drug delivery implant. In some embodiments, the implant is a gene therapy delivery implant. In some embodiments, the implant is a cell-based implant. In some embodiments, the implant is a biological tissue. In some embodiments, the implant includes a matrix or a scaffold including a natural material. In some embodiments, the implant includes a matrix or a scaffold including a synthetic material. In some embodiments, the implant includes a matrix or a scaffold including a hybrid material. In some embodiments, the implant is a RPE/fibrin hydrogel implant.

In some embodiments, the device further includes a device holder. The device holder includes a semi-open ring including a first radial arm and a second radial arm, the first and second radial arms extending from a back surface of the device holder, a first side wall and a side wall defining a guide channel, the first and second side walls extending from the back surface, and a first holder arm and a second holder arm integrally connected to the back surface, the first and second holder arms defining an opening. The opening and the guide channel are configured to receive the tip assembly.

In another aspect, the disclosure is directed to a tip assembly for implanting an implant. The tip assembly includes a hub including an actuator, a tip housing configured to house the implant, and a tube extending between the hub and the tip housing. The actuator is configured to eject the implant from the tip housing.

In some embodiments, the actuator is a spring loaded pin. In some embodiments, the spring loaded pin is in communication with a pneumatic actuator, wherein the spring loaded pin is configured to eject the implant from the tip housing upon actuation of the spring loaded pin. In some embodiments, the tube houses a guidewire extending between the actuator of the hub and the tip housing. In some embodiments, the tip housing includes a plunger coupled to the guidewire, wherein plunger is in communication with the actuator, wherein the plunger is configured to eject the implant from the tip housing upon actuation of the plunger. In some embodiments, the tip housing is clear. In some embodiments, the tube is stainless steel.

In some embodiments, the tube defines a radius of curvature. In some embodiments, the radius of curvature is about 3 mm to about 60 mm. In some embodiments, the tube includes a seal, wherein the seal is configured to inhibit eye fluid from entering the tip hub. In some embodiments, the actuator is a pneumatic actuator. In some embodiments, the pneumatic actuator is compatible with a vitrectomy unit. In some embodiments, the tip hub is configured to be removably coupled to a handle assembly. In some embodiments, the tip hub includes threads to removably couple the tip hub to the handle assembly. In some embodiments, the tip assembly is disposable. In some embodiments, the tip assembly is preloaded with the implant. In some embodiments, the implant is a drug delivery implant. In some embodiments, the implant is a gene therapy delivery implant. In some embodiments, the implant is a cell-based implant. In some embodiments, the implant is a biological tissue. In some embodiments, the implant includes a matrix or a scaffold including a natural material. In some embodiments, the implant includes a matrix or a scaffold including a synthetic material. In some embodiments, the implant includes a matrix or a scaffold including a hybrid material. In some embodiments, the implant is a RPE/fibrin hydrogel implant.

Particular embodiments of the subject matter described in this document can be implemented to realize one or more of the following advantages. First, the implantation device described herein is capable of being operated with one hand and providing actuation via a foot switch. Such a configuration is advantageous because no motion on the implantation device is needed to cause actuation. Accordingly, error of implantation is reduced as no hand or finger movement is occurring to the implantation device during ejection. Second, the curvature of the tube of the implantation device is substantially similar to an eye, such that implantation can occur in the correct location without stress on the sclera. Third, the fluid compartment of the actuator does not come into contact with fluid from the eye. Accordingly, the handle assembly can be reusable with reduced risk of contamination and adverse effects. In addition, the tip assembly can be disposable. Fourth, the tip assembly can be preloaded with an implant, reducing overall time for the procedure. This reduces handling errors and potential damage to the implant during loading. Fifth, the tip of the tip assembly can be clear, such that polarity of the implant can be determined and the implant can be implanted with the correct orientation for polarity, reducing risks associated with incorrect implantation. It also allows for screening of damaged implants prior to implantation.

The use of the term “about,” as used herein, refers to an amount that is near the stated amount by about 10%, 5%, or 1%, including increments therein. For example, “about” can mean a range including the particular value and ranging from 10% below that particular value and spanning to 10% above that particular value.

The use of the term “tube,” as used herein, refers to a hollow elongated cylindrical structure. The term “tube,” as used herein, can also refer to a hollow elongated shaft having a generally flattened oval-shaped cross section (e.g., the “delivery shaft”) in accordance with some embodiments provided herein.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described herein. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description, drawings, and from the claims.

### DESCRIPTION OF DRAWINGS

FIG. 1 is a schematic of a first implantation device for implanting RPE monolayer/fibrin implants into an eye in accordance with some embodiments provided herein.

FIG. 2 is a schematic of a second implantation device for implanting RPE monolayer/fibrin implants into an eye in accordance with some embodiments provided herein.

FIG. 3 is a cross sectional view of the second implantation device of FIG. 2 in accordance with some embodiments provided herein.

FIG. 4 is a second cross sectional view of the second implantation device of FIG. 2 in accordance with some embodiments provided herein.

FIG. 5 is a cross sectional view of a tip assembly of the second implantation device of FIG. 2 in accordance with some embodiments provided herein.

FIG. 6 is a perspective view of the tip assembly of FIG. 5 in accordance with some embodiments provided herein.

FIG. 7 is a perspective view of the tip of the tip assembly of FIG. 5 in accordance with some embodiments provided herein.

FIG. 8 is a cross sectional view of the tip of the tip assembly of FIG. 5 in accordance with some embodiments provided herein.

FIG. 9A is a perspective view of an example plug attached to a distal portion of an example implantation device in accordance with some embodiments provided herein.

FIG. 9B is an enlarged view of the example plug of FIG. 9A in accordance with some embodiments provided herein.

FIG. 10A is a perspective view of another example plug attached to a distal portion of an example implantation device in accordance with some embodiments provided herein.

FIG. 10B is an enlarged view of the example plug of FIG. 10A in accordance with some embodiments provided herein.

FIG. 11A is a perspective view of a third implantation device in accordance with some embodiments provided herein.

FIG. 11B is an enlarged view of a distal portion of the third implantation device of FIG. 11A, containing an implant in accordance with some embodiments provided herein.

FIG. 12A is an exploded view of the third implantation device of FIG. 11A in accordance with some embodiments provided herein.

FIG. 12B is a cross-sectional view of the delivery shaft in accordance with some embodiments provided herein.

FIG. 13 is a side view of a distal end of a tip of any of the implantation devices disclosed herein in accordance with some embodiments provided herein.

FIG. 14A is a perspective view of an example implantation device holder in accordance with some embodiments provided herein.

FIG. 14B is a front view of the example disposable tip holder of FIG. 14A in accordance with some embodiments provided herein.

FIG. 15A shows the third implantation device of FIG. 11A docked within the implantation device holder of FIGS. 14A and 14B in accordance with some embodiments provided herein.

FIG. 15B shows the third implantation device of FIG. 11A docked within the implantation device holder of FIGS. 14A and 14B and loaded within a container in accordance with some embodiments provided herein.

FIG. 16 is a graph showing data on intraocular pressure (IOP) maintenance with the implantation devices of the disclosure in an ex vivo porcine eye. The IOP within the porcine eye was set to 30 millimeters of mercury (mmHg). The data is reported as mean±standard deviation (n=10). “Without plug” indicates an example implantation device without a plug attached. P1, P2, P3, T1, T2, and T3 are various example prototypes of the implantation device. P1 had external dimensions of 1.41 millimeters (mm)×2.59 mm; P2 had external dimensions of 1.38 mm×2.52 mm; P3 had external dimensions of 1.44 mm×2.57 mm; T1 had external dimensions of 1.65 mm×2.92 mm; T2 had external dimensions of 1.37 mm×2.52 mm; T3 had external dimensions of 1.30 mm×2.59 mm.

Like reference numbers represent corresponding parts throughout.

### DETAILED DESCRIPTION

This document describes a stem cell-based implantation device. For example, this document relates to methods and devices for delivering iPSC-RPE cells on a fibrin scaffold into the sub-retinal space of the eye.

Macular degeneration diseases represent a variety of diseases and etiology, but commonly stem from retinal pigment epithelium (RPE) dysfunction. Genetic macular degenerations, including the bestrophinopathies, occur due to protein mutations involved in RPE function. The bestrophinopathies (e.g., Best's disease) arise from a mutation in the Best1 gene, causing RPE dysfunction leading to eventual photoreceptor death.

While the genetically-caused macular degenerations are rare, age-related macular degeneration (AMD) is the leading cause of blindness in the first world. It is estimated to account for 5 million cases in the US in 2050. AMD is a more complex disease of immune and vascular function that directly affects RPE function.

RPE replacement as a treatment for macular degeneration has been a popular focus in recent history. Modern advances in stem cell technologies have made embryonic stem cells (ESCs) and induced pluripotent stem cells (IPSCs) attractive candidates for transplantation.

Embodiments described below include implantation devices featuring actuator-loaded handles and tip assemblies designed to place cell-based implants within the eye. In some embodiments, a distinct advantage of the implantation devices described herein is they are capable of being operated with one hand (e.g., by providing actuation via a foot switch). Such a configuration is advantageous because no motion on the implantation device is needed to cause actuation. Accordingly, error of implantation is reduced as no hand or finger movement is occurring to the implantation device during ejection. In some embodiments, an additional advantage of the implantation devices described herein is that the curvature of the tube of the implantation device being substantially similar to the curvature of an eye, such that implantation can occur in the correct location without stress on the sclera. In some embodiments, yet another advantage of the implantation devices described herein is that the fluid compartment of the actuator does not come into contact with fluid from the eye. Accordingly, the handle assembly can be reusable with reduced risk of contamination and adverse effects. In addition, the tip assembly can be disposable. In some embodiments, another advantage of the implantation devices described herein is that the tip assembly can be preloaded with an implant, reducing overall time for the procedure. This reduces handling errors and potential damage to the implant during loading. In some embodiments, an additional advantage of the implantation devices described herein is that the tip of the tip assembly can be composed of a clear material, such that polarity of the implant can be determined and the implant can be implanted with the correct orientation for polarity, reducing risks associated with incorrect implantation. The clear tip of the tip assembly can also allow for screening of damaged implants prior to implantation.

Referring to the figures generally, a device can be used to place implants within an eye. The implants, in some instances, can include a cell-based implant. For example, the device can be used to place retinal pigment epithelium (RPE) monolayers within a sub-retinal space of the eye. The RPE monolayer may be supported with a scaffold that can be degradable. In other examples, the implants can be acellular implants.

Fibrin hydrogels can be used as a temporary substrate for RPE transplantation. The fibrin hydrogel can be a basal support substrate or an apically-apposed substrate for the RPE. In some cases, the RPE can be sandwiched between two fibrin hydrogels: one basal support substrate and one apically-apposed substrate.

The RPE monolayer/fibrin implants provided herein can maintain the RPE as a flat, wrinkle-free monolayer. In some cases, the fibrin configuration can provide mechanical support and protection during the transplantation process and can ensure implantation of correct RPE polarity. In some cases, the RPE monolayer/fibrin implants provided herein can reduce potential chronic inflammation, can reduce obstacles to RPE/Bruch's membrane attachment, and can maintain diffusion permeability from choroid.

A fibrin hydrogel provided herein can be easily maneuvered with surgical tools for precise orientation and location. In some cases, a fibrin hydrogel provided herein can be pliable, while maintaining its original shape and surface properties. In some cases, adherent cells do not detach from the surface of a fibrin hydrogel provided herein.

In some cases, an RPE/fibrin hydrogel implant provided herein can be produced over a collagen gel. In such cases, the RPE/fibrin hydrogel implant can be harvested using collagenase (e.g., from about 200 U/mL to about 1500 U/mL of collagenase). Collagenase does not interfere with cell-cell interaction and allows the RPE monolayer to detach from the collagen gel. The RPE monolayer also remains adhered to the fibrin hydrogel following collagenase treatment. In some cases, dispase (e.g., from about 0.5 U/mL to about 10 U/mL of dispase) can be use in addition to collagenase or in place of collagenase.

The RPE/fibrin hydrogel implant provided herein can be used to treat eye conditions such as high myopia, angioid streaks, and macular degeneration. Some of the diseases that classify as macular degeneration and that can be treated as described herein include, but are not limited to, age-related macular degeneration (AMD), central geographic atrophy, bestrophinopathies, Leber's congenital amaurosis, choroideremia, Gyrate atrophy, Sorsby's macular dystrophy, mitochondrial-inherited diabetes and deafness (MIDD), chloroquine-associated retinopathy, malattia leventinese, North Carolina dystrophy, hyperornithinemia, central serous chorioretinopathy, adult-onset foveomacular dystrophy, and Stargardt's disease.

For example, a mammal (e.g., a human) can be prepared for eye surgery, and a sub-retinal detachment is created to expose a damaged RPE region. At this point, an implantation device can be used to deliver an RPE/fibrin hydrogel implant onto the region of interest. In some cases, a cannula can be used to gain access to the eye. In some cases, an air-phase bubble may be used to push the RPE/fibrin hydrogel implant into place.

An implantation device for implanting an RPE monolayer/fibrin implant provided herein into an eye can be a plunger style device with a mechanical control of ejection. In some cases, an implantation device can be designed to deliver various sized RPE monolayer/fibrin implants and to have the ability to insert multiple implants rapidly using clip-style tips. In some cases, an implantation device provided herein can have a liquid reservoir to maintain hydration of cells and hydrogel. In some cases, an implantation device provided herein can be designed for one hand manipulation and use.

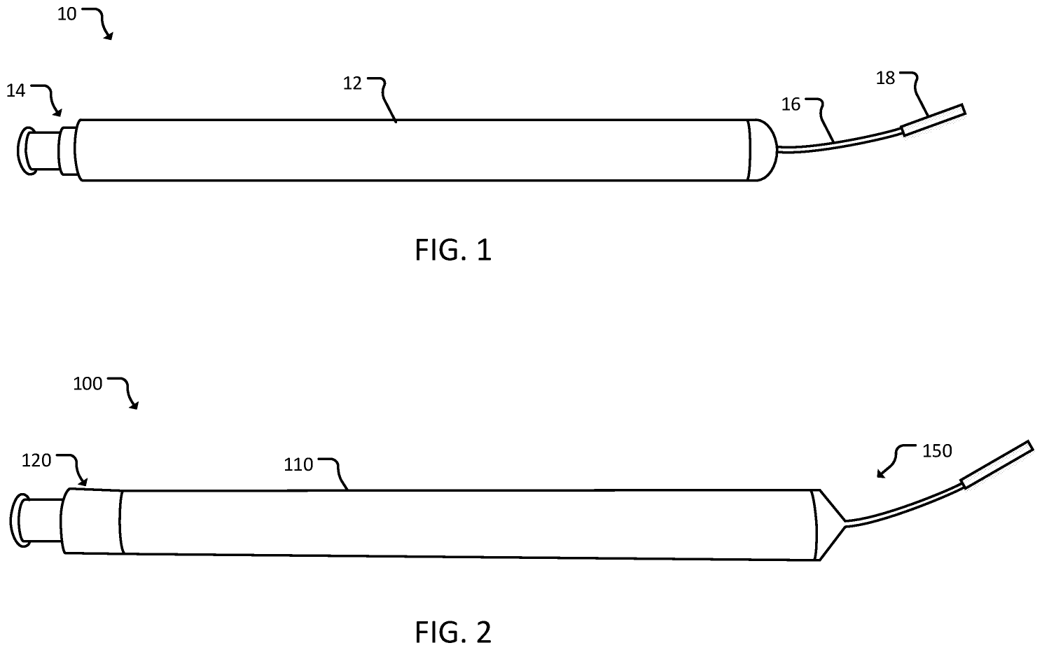

Referring to FIG. 1, a first implantation device 10 for implanting RPE monolayer/fibrin implants into an eye can include a handle portion 12, a pressure-based actuator 14, a tube 16, and a plastic tip 18.

Pressure-based actuator 14 can be driven by a liquid pneumatic, with a syringe applying the pressure. In some cases, a syringe pump or a viscous fluid injection system (e.g., of a vitrectomy system) can be used.

Plastic tip 18 can include a plunger and house a RPE monolayer/fibrin implant. In some cases, plastic tip 18 can be transparent. In some cases, plastic tip 18 can have a geometry to house an implant with a size of about 1.5 mm by about 5 mm by about 0.2 mm. In some cases, the plastic tip 18 only requires a small scleral incision for implantation of the implant.

In some cases, tube 16 can be a metal tube. Tube 16 can include a guidewire that is attached to the plunger of plastic tip 18, such that pressure is transmitted from the pressure-based actuator 14 to the plunger and causes the implant to exit the plastic tip 18. In some cases, tube 16 can match the same width and height dimensions as plastic tip 18.

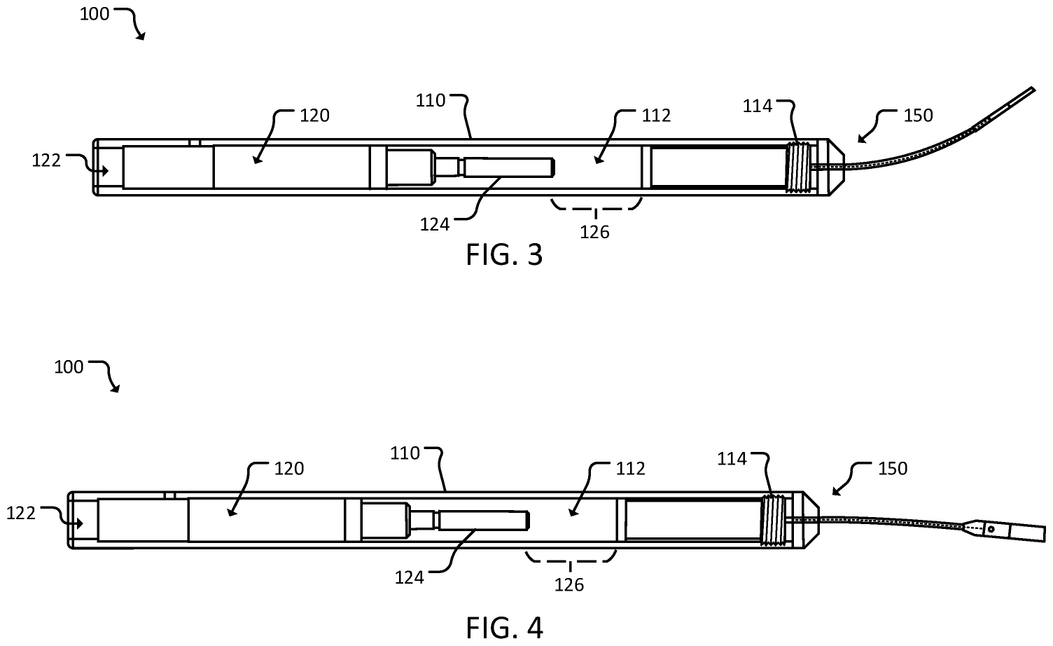

Referring to FIGS. 2-4, a second implantation device 100 for implanting RPE monolayer/fibrin implants into an eye can include a handle assembly 110 and a tip assembly 150.

Handle assembly 110 can define a lumen 112 extending along a longitudinal axis of handle assembly 110. In some cases, handle assembly 110 can include a threaded portion 114. Threaded portion 114 can be defined along a distal portion of handle assembly 110. In some cases, threaded portion 114 is defined in lumen 112. In some cases, handle assembly 110 can receive a portion of tip assembly 150. In some cases, handle assembly 110 can couple with tip assembly 150 via threaded portion 114.

Handle assembly 110 can include a pneumatic actuator 120 within lumen 112. Pneumatic actuator 120 can include a pneumatic fluid inlet luer 122. In some cases, pneumatic actuator 120 can include an actuator piston 124. In some cases, pneumatic actuator 120 can be capable of using fluid pressure to actuate implantation device 100 and deploy an implant. In some cases, pneumatic actuator 120 can be compatible with vitrectomy units for a surgeon to deploy the implant using a vitrectomy unit foot switch mechanism for hands-free actuation. In some cases, the fluid from the pneumatic actuator 120 does not come into contact with tip assembly 150. In some cases, handle assembly 110 can be reusable.

In some cases, when in an unactuated state, a gap 126 is defined between actuator piston 124 and tip assembly 150. In some cases, gap 126 can be a safety feature of implantation device 100. For example, gap 126 can ensure an implant is not expelled from tip assembly 150 when attaching tip assembly 150 to handle assembly 110 by ensuring actuator piston 124 does not come into contact with tip assembly 150 before pneumatic pressure is applied. Accordingly, gap 126 can aid in preventing the implant from being ejected prematurely.

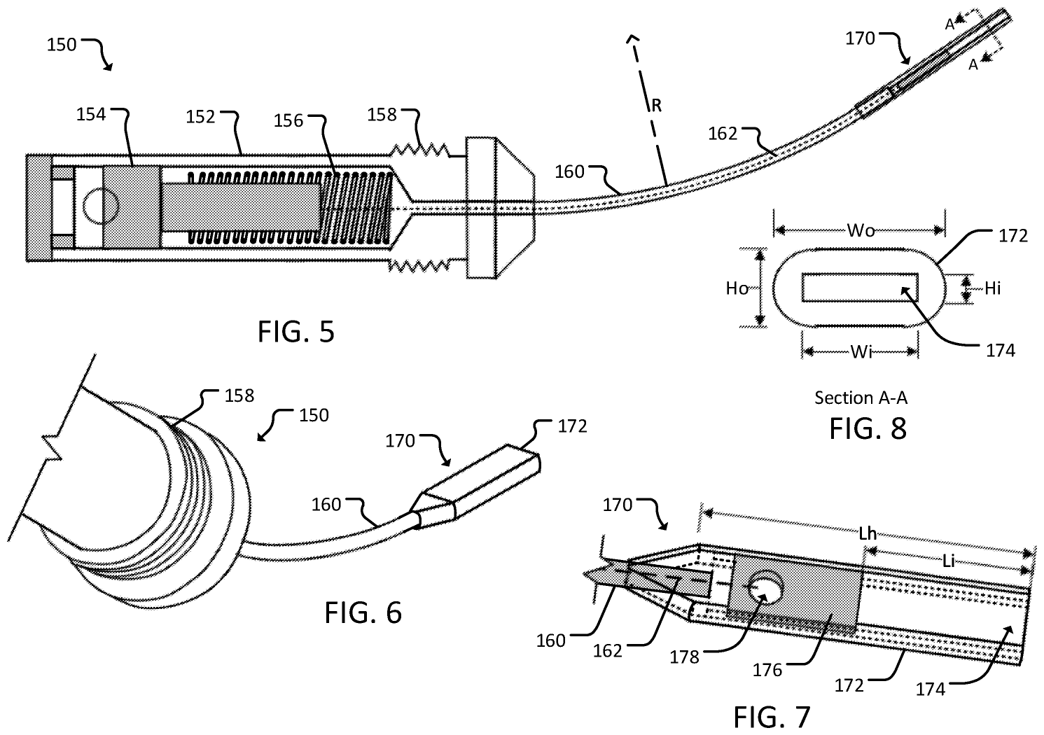

Referring to FIGS. 5-8, a tip assembly 150 of the implantation device 100 can include a hub 152, a tube 160, and a tip 170. Hub 152 can houses a pin 154 and a spring 156. In some cases, hub 152 can include threads 158. In some cases, hub 152 can be plastic. In some cases, hub 152 can be clear, transparent, opaque, or solid.

In some cases, tip assembly 150 can be preloaded with an implant in the tip 170. In some cases, when tip assembly 150 is preloaded with an implant, tip assembly 150 and/or tip 170 can be shipped in a liquid containing vessel to keeps implant cells alive and protect from damage. In some cases, the liquid can be a culture medium. In some cases, tip assembly 150 can be disposable.

Pin 154 can be actuated by actuator piston 124. In some cases, actuator piston 124 comes into contact with pin 154 to provide actuation. Spring 156 can provide resistance of actuation of pin 154. In some cases, the resistance of spring 156 aids in ensuring adequate pressure is applied before the implant is ejected. In some cases, spring 156 can cause pin 154 to move back to a neutral position once pressure is no longer applied.

Threads 158 can be located on an exterior surface of hub 152. In some cases, threads 158 can allow tip assembly 150 to be coupled to handle assembly 110. In some cases, threads 158 can be complementary to threads 114.

In some cases, tube 160 is metal. For example, tube 160 can be stainless steel. Tube 160 can pass a guidewire 162 from hub 152 to tip 170. Guidewire 162 can be coupled to a distal end of pin 154, such that actuation of pin 154 causes actuation of guidewire 162. In some cases, tube 160 can have a radius of curvature R. In some cases, the radius of curvature R is about 3 mm to about 60 mm. In some cases, the radius of curvature R can be substantially similar to a radius of curvature of an eye, such that when implanting the implant, tip 170 can move parallel to the interior surface of the posterior eyecup. In some cases, the radius of curvature R can aid in achieving the correct location for implantation without straining the sclera, choroid, or retina or contacting the inner retinal surface. In some cases, tube 160 can include a seal. In some cases, the seal can aid in preventing eye fluid from entering hub 152 and/or handle assembly 110. In some cases, the seal can aid in maintaining separation between the fluid for actuation and the eye fluid. In some cases the seal can prevent pressure dependent fluid flow into the tip 170 and allow the implant to come out of the tip 170.

Tip 170 can include a tip housing 172 and a plunger 176. Tip housing 172 can define an inner space 174. Inner space 174 can receive an implant. In some cases, the implant is an RPE/fibrin hydrogel. In some cases, the implant can have a size of about 1.0 mm by about 6.0 mm by about 0.05 mm to about 6.0 mm by 6.0 mm by 0.4 mm. In some cases, tip housing 172 is clear, transparent, or partially transparent, such that the inner space 174 can be visualized. In some cases, the implant has a polarity. In some cases, the polarity needs to be correct during implantation. In some cases, the polarity is different between the top and the bottom of the implant. In some cases, a size of the implant can dictate a size of tip housing 172, such that different sizing of tip housing 172 are used for different implants.

Tip housing 172 has a tip housing length Lh. In some cases, tip housing length Lh is a length of a rectangular portion of tip housing 172. In some cases, tip housing length Lh can be about 5 mm to about 15 mm. Tip housing 172 has a tip housing implant length Li that traverses the inner space 174. In some cases, tip housing implant length Li is a length of the rectangular portion of tip housing 172 that can house an implant. In some cases, tip housing implant length Li can be about 5 mm to about 8 mm. As shown in FIG. 8, yip housing 172 has a tip housing outer width Wo. In some cases, tip housing outer width Wo is a width of the rectangular portion of tip housing 172. In some cases, tip housing outer width Wo can be about 0.5 mm to about 6.2 mm. In some cases, tip housing outer width Wo can be about 1.6 mm to about 2.9 mm. Tip housing 172 has a tip housing outer height Ho. In some cases, tip housing outer height Ho is a height of the rectangular portion of tip housing 172. In some cases, tip housing outer height Ho can be about 0.05 mm to about 2 mm. In some cases, tip housing outer height Ho can be about 0.3 mm to about 1.3 mm.

Tip housing 172 has a tip housing inner width Wi. In some cases, tip housing inner width Wi is a width of inner space 174 of the rectangular portion of tip housing 172. In some cases, tip housing inner width Wi can be about 0.5 mm to about 1.8 mm. Tip housing 172 has a tip housing inner height Hi. In some cases, tip housing inner height Hi is a height of inner space 174 of the rectangular portion of tip housing 172. In some cases, tip housing inner height Hi can be about 0.1 mm to about 0.7 mm.

In some cases, plunger 176 can be plastic. Plunger 176 can actuate to eject the implant. In some cases, plunger 176 defines an aperture 178. In some cases, aperture 178 is attached to guidewire 162, such that actuation of guidewire 162 causes actuation of plunger 176. In some cases, plunger 176 can be a stainless steel bent wire. In some cases, plunger 176 can be an extension of the guidewire 162. In some cases, the guidewire can be a flat plastic strip that becomes plunger 176.

When handle assembly 110 is actuated (e.g., via pneumatic actuator 120), actuator piston 124 moves forward and pushes on pin 154, compressing spring 156. As pin 154 moves, guidewire 162 moves forward, pushing plunger 176 forward, causing the implant to be ejected from the tip assembly 150. In some cases, once pneumatic pressure is eliminated, spring 156 causes pin 154, guidewire 162, and plunger 176 to return to a neutral position.

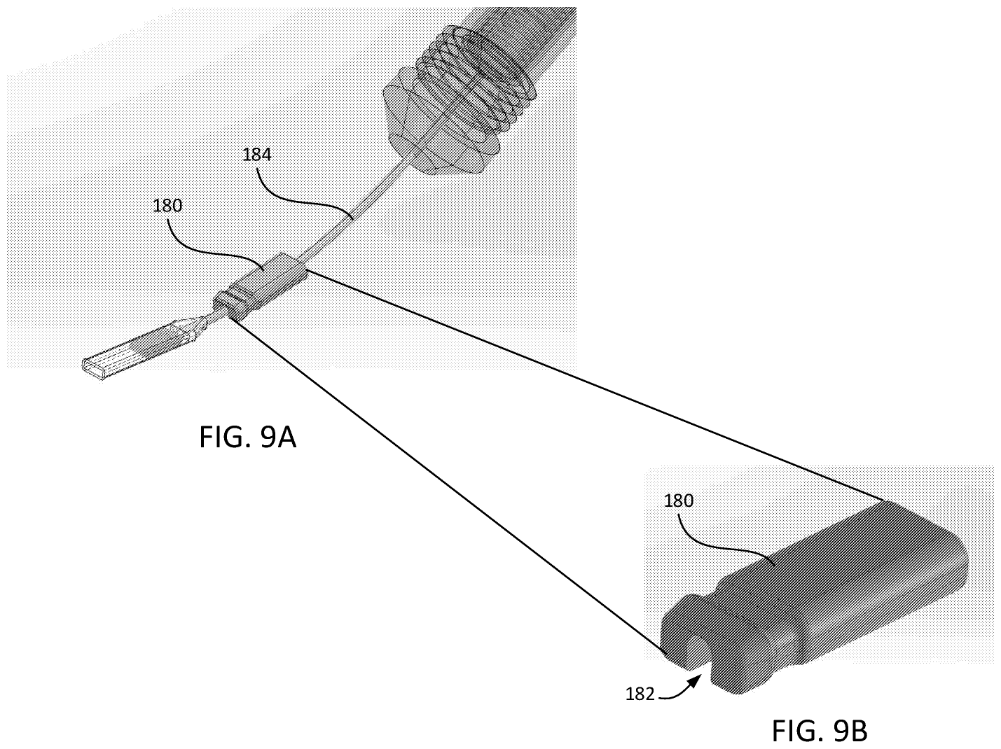

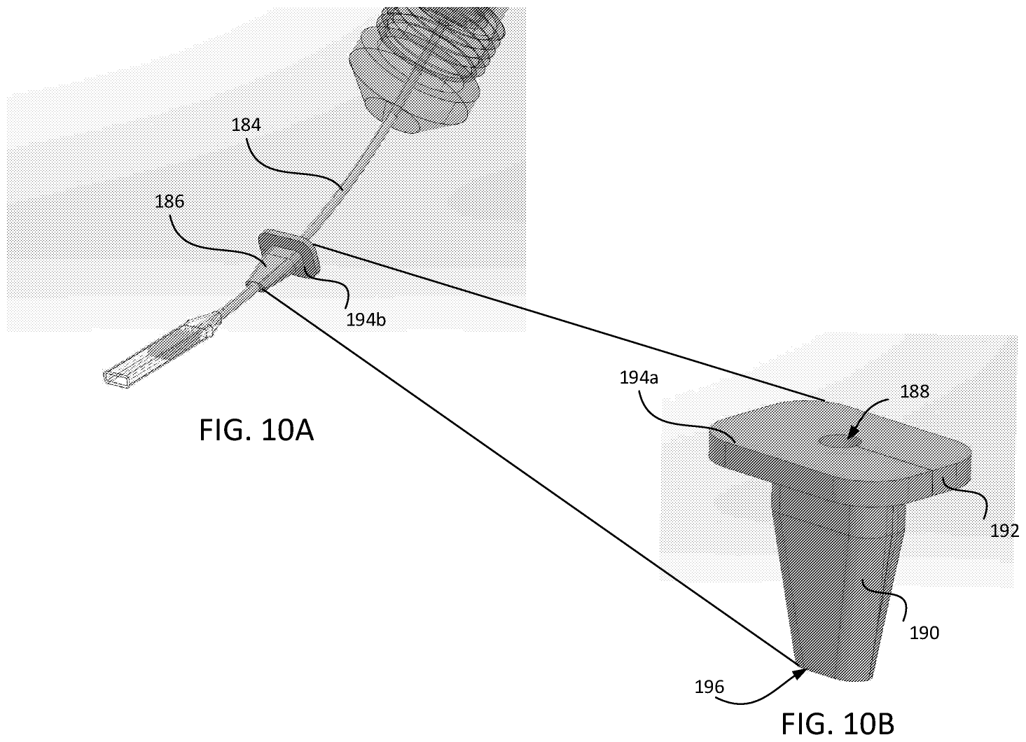

Referring briefly to FIGS. 9A, 9B, 10A, and 10B, any of the implantation devices of the disclosure can include a plug. The two different plug designs presented in FIGS. 9A, 9B, 10A, and 10B can help maintain normal or adequate intraocular pressure (IOP) of an individual undergoing an ocular implantation procedure as described herein. For example, by plugging a potential difference in width of a scleral incision, which is done during the ocular implantation procedure (e.g., during a sclerotomy), and a width of the tube (e.g., tube 160), the plugs of the disclosure can help maintain appropriate IOP. In some cases, the plug can be a snap-on attachment that docks with the tube of an implantation device of the disclosure. The two different plug designs presented in FIGS. 9A, 9B, 10A, and 10B can be configured to slide along the tube so that the plug can adjustably attached to the tube (e.g., upon a user exerting a force onto the plug to slide it along the tube) and remain in place (e.g., when the plug is left untouched) while still allowing the tip of the implantation device (e.g., tip 170) to line up properly during a retinotomy.

FIGS. 9A and 9B show a distal portion of an example implantation device including a first plug 180. Any of the implantation devices disclosed herein can include a first plug 180. In some cases, first plug 180 is attached (e.g., reversibly attached or permanently attached) to the implantation device. In some cases, first plug 180 is integrally mounted to the implantation device. In some cases, the first plug 180 can be three-dimensionally (3D) printed with a polymer filament (e.g., a poly-lactic acid (PLA) filament). However, in other cases, first plug 180 can be fabricated by other methods known to the skilled artisan. Once 3D printed, the edges of the first plug 180 can be smoothed or polished (e.g., with a sharp object such as a scalpel) to generate a smooth surface and/or edges. First plug 180 can have a rectangular shape defining a slot 182 configured to receive tube 184. In some cases, slot 182 has a width that is sufficiently greater than an outer width of the tube (e.g., tube 184) such that it provides a snap-fit engagement between the first plug 180 and the tube (e.g., tube 184). In some cases, plug 180 includes a channel through which tube 184 can be threaded through.

FIGS. 10A and 10B show a distal portion of an example implantation device including a second plug 186. Any of the implantation devices disclosed herein can include a second plug 186. In some cases, second plug 186 is attached (e.g., reversibly attached or permanently attached) to the implantation device. In some cases, second plug 186 is integrally mounted to the implantation device. In some cases, second plug 186 is snapped onto the tube 184. Second plug 186 can have a body 190 integrally attached to a plate 192. In some cases, body 190 has a tapered shape, as shown in FIG. 10B. In some cases, plate 192 is a rectangular plate having a first surface 194a and a bottom surface 194b. In some cases, bottom surface 194b can contact an ocular surface of a patient during an implantation procedure. Second plug 192 can define a channel 188 that extends from the top surface 194a of the plate 192 through the distal end 196 of the body 190. Channel 188 can be configured to receive tube 184. In some cases, channel 188 has a diameter that is sufficiently wider than the outer diameter of the tube (e.g., tube 184) such that it enables second plug 186 to be slid along the tube (e.g., upon a user exerting a force onto the plug to slide it along the tube) and to remain in place (e.g., when the plug is left untouched).

In some cases, the second plug 186 can be fabricated by generating a “negative” mold via 3D printing. However, in other cases, the “negative” mold can be fabricated by other methods known to the skilled artisan. Example materials used to 3D print the “negative” mold include, but are not limited to, PLA, polystyrene, polyvinyl acetate, polyamide, polyethylene, polypropylene, polycarbonate, polyoxymethylene (POM) and epoxy resins. In some cases, second plug 186 is composed of polydimethylsiloxane (PDMS). In some cases, to fabricate second plug 186 composed of a silicone elastomer (e.g., PDMS), the silicone elastomer is prepared per manufacturer's protocol and degassed under vacuum for about 30 minutes. A silicone-releasing agent can be sprayed within the 3D-printed, “negative” mold. Next, the “negative” mold can then be filled with the silicone elastomer. Once the mold is filled, a 23 gauge needle can be inserted through the center of the mold to create channel 188. Next, second plug 186 (i.e., the mold filled with the silicone elastomer at this stage) can be cured at about 100° C. for about 2 hours. Once cooled down, the second plug 186 can be removed from the mold, and a slit can be cut lengthwise on the side of body 190 to enable a user to reversibly attach the second plug 186 onto the tube.

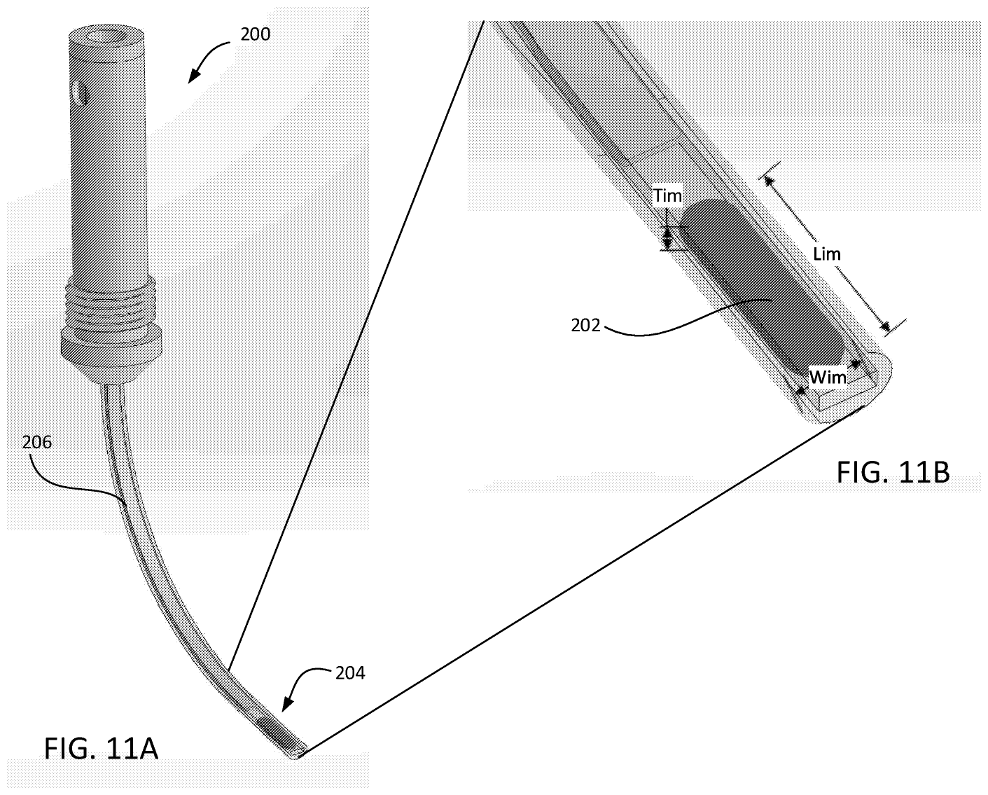

An implantation device may be substantially similar in construction and function in several aspects to the first implantation device 10 and second implantation device 100 discussed above, but can include an alternative delivery mechanism instead of tubes 16 and 160 and guidewire 162, for example. FIGS. 11A and 11B show a third implantation device 200 including an implant 202 located within a flat tip 204 of the device. Third implantation device 200 includes a delivery shaft 206 with a smooth, seamless width that can help maintain IOP during the surgery and reduce complications associated with fluctuating IOP. In some cases, implant 202 can have a length Lim of about 5 millimeters (mm). In some cases, implant 202 can have a length Lim ranging from about 1 mm to about 10 mm. In some cases, implant 202 can have a width Wim of about 1.5 mm. In some cases, implant 202 can have a width Wim ranging from about 0.5 mm to about 6 mm. In some cases, implant 202 can have a thickness Tim of about 0.2 mm. In some cases, implant 202 can have a thickness Tim ranging from about 0.001 mm to about 1 mm.

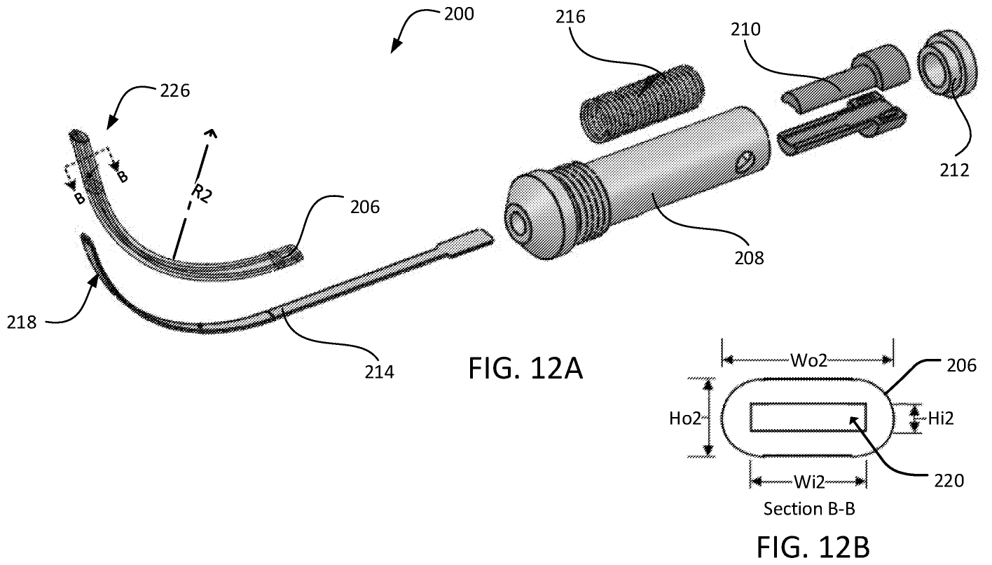

Referring to FIG. 12A, third implantation device 200 includes delivery shaft 206. In some cases, delivery shaft 206 is fabricated via an extrusion method. In some cases, delivery shaft 206 is fabricated via injection molding or micromolding. However, in other cases, delivery shaft 206 can be fabricated by other methods known to the skilled artisan. Third implantation device 200 further includes a hub 208, a pin 210, a cap 212, a push rod 214, and a spring 216. In some cases, hub 208, pin 210, and spring 216 are substantially similar in construction and function to the hub 152, pin 154, and spring 156, respectively, discussed above. In this embodiment, delivery shaft 206 can be composed of a nylon material. In some cases, delivery shaft 206 provides a gradual mechanical rigidity and an optically clear cavity to visualize the implant. Similar to the previously described embodiments, the pin 210, cap 212, and spring 216 are components of the plunger mechanism that causes the implant to be ejected from the tip assembly of the implantation device. In this embodiment, the push rod 214 is a flat rod that has a curvature that matches the curvature of the delivery shaft 206. Delivery shaft 206 is a hollow shaft having a generally flattened oval-shaped cross section and shaped such that it is configured to receive push rod 214. In other words, the cavity formed by the inner walls of delivery shaft 206 define a volume that is sufficiently large to accommodate push rod 214 within it. Delivery shaft 206 can extend from hub 208. In some cases, delivery shaft 206 can be a hollow cylindrical shaft. Push rod 214 is a solid rod having a generally flattened oval-shaped cross section. In other cases, push rod 214 can be a cylindrical rod. Push rod 214 can extend from hub 208 to the tip 226 of delivery shaft 226. In some cases, push rod 214 can extend from hub 208 to the tip housing 172.

In some cases, push rod 214 is composed of a stainless steel material. The distal tip portion 218 of the push rod can be coated with a hydrophobic material (e.g., polytetrafluoroethylene (PTFE), polyoxymethylene (POM)) in order to create a water-tight seal and prevent backflow within the delivery shaft 206 once placed in the pressurized eye of a patient. In addition, the distal tip portion 218 of the push rod 214 can be coated with a hydrophobic material in order to prevent contact of the implant with the remaining portion of the push rod 214.

When third implantation device 200 is actuated (e.g., via a pneumatic actuator), an actuator piston moves forward and pushes on pin 210, compressing spring 216. As pin 210 moves, push rod 214 moves forward, pushing the implant forward, causing the implant to be ejected from the flat tip 204. In some cases, once pneumatic pressure is eliminated, spring 216 causes pin 210, and push rod 214 to return to a neutral position.

As shown in FIG. 12B, delivery shaft 206 has an outer width Wo2. In some cases, delivery shaft outer width Wo2 can be about 2.5 mm. In some cases, delivery shaft outer width Wo2 can be about 1.0 mm to about 7.0 mm. Delivery shaft 206 has an outer height Ho2. In some cases, delivery shaft outer height Ho2 can be about 1.5 mm. In some cases, delivery shaft outer height Ho2 can be about 0.1 mm to about 2.5 mm.

Delivery shaft 206 has a delivery shaft inner width Wi2. In some cases, delivery shaft inner width Wi2 can be about 1.6 mm. In some cases, delivery shaft inner width Wi2 can be about 0.5 mm to about 6.1 mm. Delivery shaft 206 has a delivery shaft inner height Hi2. In some cases, delivery shaft inner height Hi2 can be about 0.4 mm. In some cases, delivery shaft inner height Hi2 can be about 0.005 mm to about 1.5 mm. Delivery shaft inner width Wi2 and inner height Hi2 define cavity 220. In some cases, cavity 220 is configured to receive push rod 214.

FIG. 12A shows a curvature R2 of the delivery shaft 206. Similar to the previously described embodiments, a radius of curvature of the delivery shaft 206 can help align the implant during the retinotomy for correct insertion. In some cases, the radius of curvature R2 of the delivery shaft 206 can range from about 20 mm to about 50 mm.

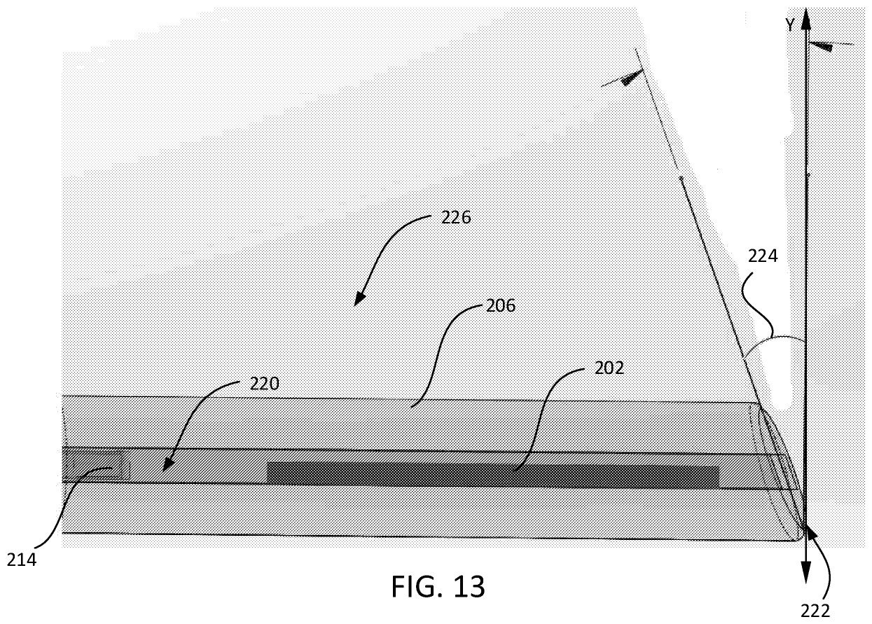

Referring to FIG. 13, the implantation devices of the disclosure can have a beveled end 222. The tip 226 of delivery shaft 206 can have a beveled end 222. Beveled end 222 can help guide surgical placement of the implantation device. A bevel angle 224, formed with respect to the Y-axis (and with respect to cavity 220) is shown in FIG. 13. In some cases, bevel angle 224 can be about +20°. In some cases, bevel angle 224 can be about −40° to about 0° to about +40°.

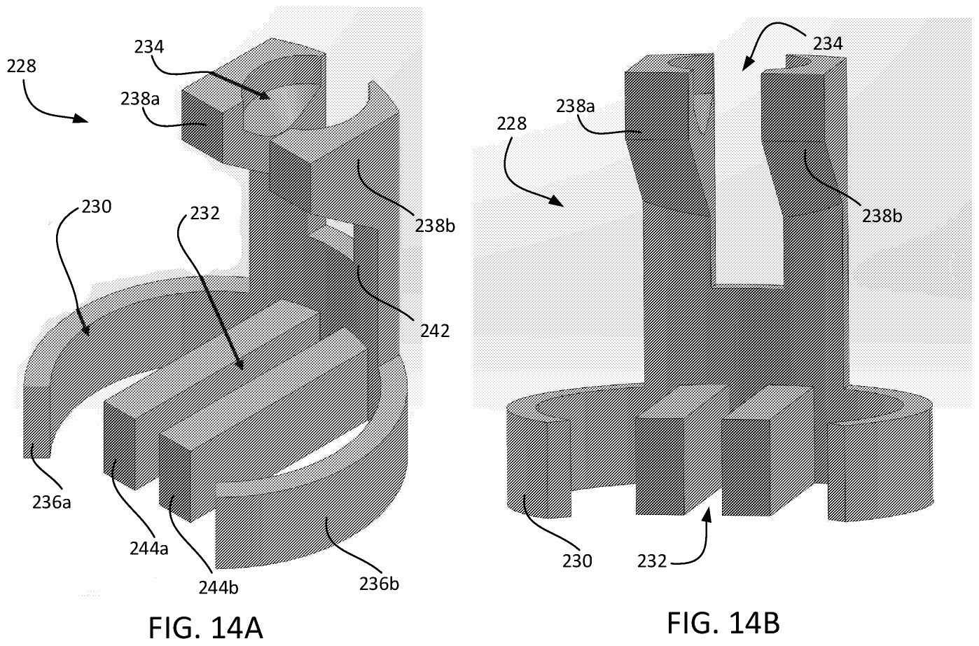

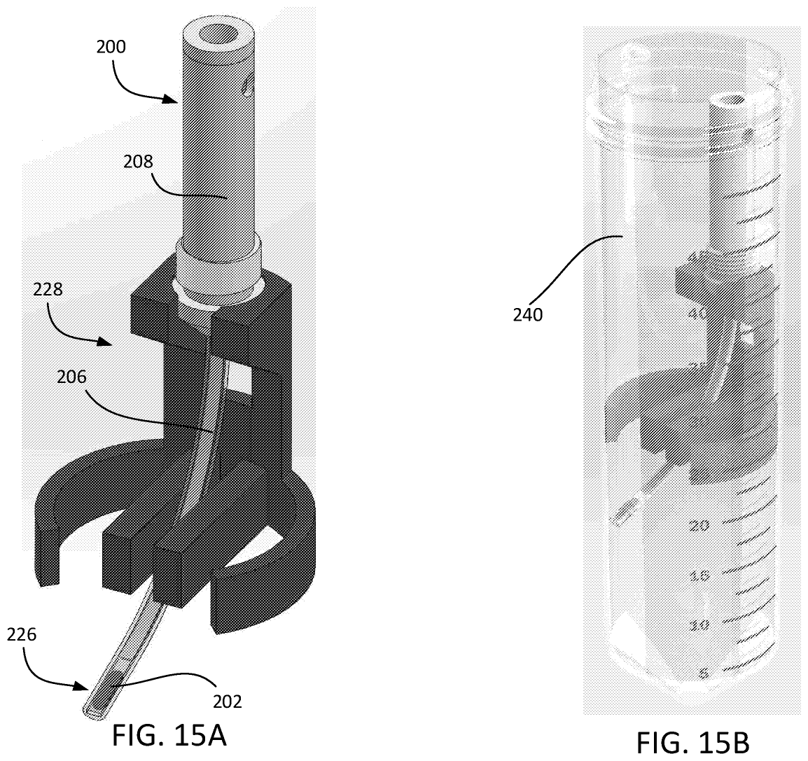

In some embodiments, implants can be pre-packaged within a disposable tip or delivery shaft. FIGS. 14A and 14B show a holder 228 designed to hold a disposable tip or delivery shaft including an implant. FIG. 15A shows the third implantation device 200 secured by the holder 228. FIG. 15B shows the third implantation device 200 secured by the holder 228 inside a storage container 240.

In some cases, the implant requires a cell culture medium with a sufficient volume necessary to sustain viability of the cells disposed on and/or within the implant and/or to increase a shelf life of the implant. In some cases, the sufficient cell culture medium volume ranges from about 1 milliliters (mL) to about 50 mL. In some cases, the tip and/or tube or delivery shaft of an implantation device is stored in a storage container. In some cases, the storage container is a Good Manufacturing Practice (GMP)-qualified container. In some embodiments, the storage container maintains biocompatibility with the implant and compatibility with automated handling systems. In some cases, the storage container is a centrifuge tube. In some cases, the storage container is a 50 mL centrifuge tube. In some cases, the storage container is optically clear, biocompatible, and can easily be compatible with automated handling systems.

Holder 228 can be fabricated via 3D printing methods, injection molding or other methods that are known to the skilled artisan. As shown in FIG. 14A, the holder 228 includes a back surface 242, a support ring 230, a guide channel 232, and an opening 234. The support ring 230 can be a semi-open ring that includes a first and second radial arms 236a and 236b, respectively. The first and second radial arms 236a and 236b can extend from the back surface 242 of the holder 228. The support ring 230 can be designed to have some range of pliability such that the first and second radial arms 236a and 236b are flexible radially inwards when force is exerted onto the outer surfaces of first and second radial arms 236a and 236b (e.g., when a user squeezes one or both of the first and second radial arms 236a and 236b radially inward towards guide channel 232). In some cases, the support ring 230 has an outer diameter that can be slightly larger than the diameter of the storage container 236 (e.g., a 50 mL centrifuge tube). Thus, in some cases, compression is required to place the holder 228 within the storage container 236, creating a tighter hold and reducing the potential of slippage. In some cases, first and second radial arms 236a and 236b can be flexed inward to place holder 228 within a storage container 236.

Referring to FIG. 14A, holder 228 can include a first side wall 244a and a second side wall 244b that define guide channel 232. First and second side walls 244a and 244b can extend from back surface 242. Guide channel 232 can support the delivery shaft 206, which is integrally connected to the tip 226 of third implantation device 200, in order to prevent it from rotating and/or moving.

Holder 228 can include a first holder arm 238a and a second holder arm 238b, which can be integrally connected to back surface 242. The first holder arm 238a and the second holder arm 238b define an opening 234. The opening 234 can be configured to receive the hub of the implantation device. The opening 234 can be designed to have some range of pliability such that the first and second holder arms 238a and 238b, respectively, are flexible radially outwards when, for example, force is exerted onto the inner surfaces of first and second holder arms 238a and 238b. In some cases, implantation device 300 can be snapped into opening 234 (e.g., by inserting implantation device 300 in between first and second holder arms 238a and 238b). In some cases, first and second holder arms 238a and 238b and support the weight of implantation device 300 and its volumetric contents. In some cases, first and second holder arms 238a and 238b prevent movement of the tip once snapped in. In some cases, the implantation device 300 can be snapped into opening 234 such that the threads on hub 208 are accessible from above. The tension can be sufficient to allow screwing a handle 110 into the hub 208 without the need to touch the implantation device 300 or remove the holder 228 from inside the storage container 240.

When in use, the hub 208 of implantation device 200 can be snapped into the holder 228 by squeezing the hub 208 in between the first and second holder arms 238a and 238b and into opening 234. Next, the delivery shaft 206 and tip 226 can be placed within the guide channel 232. Finally, the holder 228 supporting the implantation device 200 can be placed within the storage container 240 by squeezing first and second radial arms 236a and 236b to temporarily decrease the diameter of the support ring 230 such that the temporary diameter is slightly less than the diameter of the storage container 240. Upon release of the first and second radial arms 236a and 236b, the diameter of the support ring 230 reverts to its normal size (i.e., slightly greater than the diameter of the storage container 240) and the holder 228 is held in place within the storage container 240. Holder 228 can be used with any of the implantation devices disclosed herein.

In some cases, a user performs the steps described above. In some cases, the tip is to be pre-sterilized, loaded with the implant, and placed into the storage container without the need to touch any part of the disposable tip. In some embodiments, such pre-sterilized system is compatible with GMP aseptic techniques. For example, the holder can be docked to a loading tray that allows for hands-free loading of the implant. In some cases, the holder can be held or moved with forceps as necessary. In some cases, the holder can be placed onto a loading tray such that the delivery shaft lines up with a loading channel. The dimensions of the loading channel can align with the width of the delivery shaft. The edge of the delivery shaft can align with a raised ledge, where the ledge height can be similar to the delivery shaft wall thickness. The implant can be pre-placed in this ledge within the channel and can slide or be aspirated into the delivery shaft with a spatula, forcep, or other tool seamlessly.

In some examples, the implantation devices of the disclosure can be used for the placement of implants (e.g., a tissue implant, a tissue transplant, a medical device implant, a tissue engineered implant, a soft tissue, a hard tissue, or any combinations thereof) in various spaces in the eye, orbit, or rest of a body. In some examples, the implant can be a drug delivery implant, a gene therapy delivery implant, a cell-based implant, a biological tissue, or any combinations thereof.

In some examples, the implantation devices of the disclosure can be used for the placement of fibrin hydrogel-based implants (e.g., a tissue implant) in various spaces in the eye, orbit, or rest of a body. In some cases, the implantation devices of the disclosure can be used for placement of engineered implants (e.g., a tissue implant) in various spaces in the eye, orbit, or rest of a body. In some cases, engineered implants (e.g., a tissue implant) can include a natural material, synthetic material, hybrid material (e.g., a combination of one or more natural and/or synthetic materials), or any combination thereof. In some embodiments, implants (e.g., a tissue implant) can be a scaffold including any of the implant materials disclosed herein. In some embodiments, implants (e.g., a tissue implant) can be a matrix including any of the implant materials disclosed herein. In some cases, a natural material can include, but is not limited to, alginate, collagen, gelatin, laminin, fibrin, decellularized matrix, recombinant peptides, synthetic peptides, proteins, or any combinations thereof. In some cases, a synthetic material can include, but is not limited to, polyethylene glycol (PEG), poly lactic acid (PLA), poly glycolic acid (PGA), poly lactic-glycolic acid (PLGA), poly caprolactone (PCL), polydimethylsiloxane (PDMS), poly ethylene terephthalate (PET), parylene, polycarbonate, polystyrene, polymethylmethacrylate (PMMA), polyethylene, polytetrafluoroethylene (PTFE), polyoxymethylene (POM), or any combinations thereof.

In some embodiments, the implants can include an agent. Non-limiting examples of agents include a virus, a gene delivery vector, a nanoparticle, a microparticle, a therapeutic agent, a cell, an exosome, a portion of a cell, a growth factor, a protein, a peptide, a pharmaceutical agent, a preservative, an antibacterial agent, an antiviral agent, an anti-inflammatory agent, an anesthetic, a disintegrating agent, a controlled release agent, an antifungal agent, an antiandrogenic agent, or any combination thereof.

In some cases, the implantation devices of the disclosure can be used for placement of autologously excised tissue in various spaces in the eye, orbit, or rest of a body. In some cases, the implantation devices of the disclosure can be used for placement of RPE in a sub-retinal space. In some cases, the implantation devices of the disclosure can be used for placement of photoreceptors in a sub-retinal space. In some cases, the implantation devices of the disclosure can be used for placement of neural retinal tissue. In some cases, the implantation devices of the disclosure can be used for placement of tissue in vitreous, subretinal space, suprachoroidal space, anterior chamber, schlems canal, anterior capsule, corneal surface, conjunctiva, sub-tenon, pen-orbital spaces, or any combination thereof. In some cases, the implantation devices of the disclosure can be used for cell delivery, cell-mediated trophic factor delivery, drug delivery, gene therapy delivery, or any combinations thereof to tissues in vitreous, subretinal space, suprachoroidal space, anterior chamber, schlems canal, anterior capsule, corneal surface, conjunctiva, sub-tenon, pen-orbital spaces, or any combination thereof. In some cases, the implantation devices of the disclosure can be used for placement of tissue in sub-cutaneous space, bladder, or other hydrated, soft tissues. In some cases, the implantation devices of the disclosure can be used for cell delivery, cell-mediated trophic factor delivery, drug delivery, gene therapy delivery, or any combinations thereof to tissues in a sub-cutaneous space, bladder, or other hydrated, soft tissues.

### EXAMPLES

The invention is further described in the following examples, which do not limit the scope of the invention described in the claims.

### Example 1—Protocol for Retinal Pigment Epithelium Monolayer with Basal Fibrin Support

A mixed solution of 30 mg/mL fibrinogen and 100 U/mL thrombin was plated in order to gel. For example, about 20-80 mg/mL fibrinogen was mixed with about 10-600 U/mL thrombin. The plate was swirled to ensure a uniform spread of the mixed solution. A mold was used to compress the gel to a desired thickness. For example, the desired thickness ranged between about 50 micrometers (μm) to 1 mm. The mixture was plated onto tissue culture polystyrene (TCPS). Alternatively, the mixture can also be plated onto polycarbonate and/or cellulose ester. Alternatively, flat sheets of fibrin gel can be pre-formed using a mold and mounted onto a cell culture insert. The mixture was sprayed onto the TCPS surface.

In some embodiments, the gel surface can be coated with a 1:5 dilution of Matrigel®. In some examples the dilution ratio of Matrigel® can range between about 1:1-1:50. In some examples, the gel surface can be coated with Matrigel®, geltrex, Laminin 521, Laminin 511, or a combination thereof. In other examples, coating the gel surface is not necessary.

Cells were plated at a concentration of about 0.5×10⁶ cells/centimeter squared (cm²). In some embodiments, the cell can be plated at a cell concentration ranging from about 0.1×10⁶ to about 2.0×10⁶ cells/cm².

Cells were cultured for about 2 weeks with cell culture medium containing about 50 units (U)/mL of aprotinin. In some embodiments, the cell culture medium can include aprotinin at a concentration ranging from about 20 to about 150 U/mL. In some examples, the cells can be cultured for a period of time ranging from about 1 week to about 10 weeks.

Fibrin-RPE was mobilized by peeling fibrin from the support. In some examples, plasminogen was loaded into basal fibrin gel. Next, fibrin-RPE was incubated in the plasminogen solution. The plasminogen solution ranged from about 0.001-40 U/mL (e.g., 1-40 U/mL). The fibrin-RPE was incubated for about 2-6 hours.

In other examples, apical gel was added for additional support. Briefly, about 80 μL of a mixed solution including about 50 mg/mL fibrinogen, about 2 U/mL plasminogen, and about 100 U/mL thrombin was sprayed at total flow rate of about 80 μL/second, about 0.8 bar for about 1 second at a height of about 10 cm. In some examples, about 30-200 μL of mixed solution can be sprayed. In some examples, about 30-70 mg/mL of fibrinogen can be added to the mixed solution. In some examples, about 0.1-4.0 U/mL of plasminogen can be added to the mixed solution. In some examples, about 10-600 U/mL of thrombin can be added to the mixed solution. In some examples, the mixed solution can be sprayed at about 30-400 μL/second flow rate. In some examples, the mixed solution can be sprayed at a pressure of about 0.6-1.2 bar. In some examples, the mixed solution can be sprayed to coat a total height of about 5-15 centimeters (cm).

Fibrinogen was allowed to gel for 1 hour at a temperature of about 37° C. In some examples fibrinogen can be allowed to gel for about 30 minutes to about 2 hours.

Fibrin-RPE implant was transferred to flat surface and multiple implants were punched out. The implants were loaded into an example implantation device of the disclosure. The eye was prepared for surgery. The implant was plunged into a subretinal space using an example implantation device of the disclosure. Laser tacked. Multiple implants were tiled within a subretinal space. The eye was closed.

After 24 hours, an intravitreal injection of about 100 μL of 4,000 U/mL tissue plasminogen activator was administered. In some examples, an intravitreal injection of tissue plasminogen activator can be given at about 3-72 hours post-surgery. In some examples, the intravitreal injection of tissue plasminogen activator can have a volume of about 50-200 μL. In some examples, the intravitreal injection of tissue plasminogen activator can have a concentration of about 100-35,000 U/mL.

This protocol produced an RPE monolayer with basal fibrin support.

### Example 2: Intraocular Pressure Measurements in a Cadaver Pit Eve

This protocol represents a means to assess maintenance of intraocular pressure (IOP) during an ocular technique requiring a sclerotomy. The method was developed to assess the ability of the various devices with or without plugs to maintain the IOP during device usage.

IOP measurement experiments were performed with ten cadaver pig eyes. Freshly enucleated pig eyes were provided by the Hormel Institute (Austin, Minn.), and the experiment was scheduled within 6 hours from the tissue obtainment. The eyes were prepared under routine surgical setup. Four 25-gauge trocars (Alcon) were placed at 3.5 mm far from the limbus. The fourth port was used for 25-gauge chandelier illumination (Alcon). To enable visualization, a pars plana lensectomy was performed. A core vitrectomy was performed using the Accurus® Surgical System. The Zeiss Opmi 150 Microscope (Zeiss, Germany) was used with the Oculus Binocular Indirect Ophthalmic Microscope wide-angle visual system (BIOM System; Oculus, FL USA) to visualize the retina and vitreous. Staining of the posterior hyaloid was performed using up to 0.5 ml of triamcinolone acetonide (Triesence® 40 mg/ml, Alcon) followed by posterior hyaloid detachment until no residual vitreous was confirmed on the retinal surface. Vitrectomy was also performed near the site of the sclerotomy in order to limit impeding the opening. The vitrectomy cutter was used to create a retinal detachment and a 1.6 mm retinotomy adjacent to the area centralis of the pig retina. A sclerotomy was performed with a 3.6 mm width, to allow access to the intraocular space for the various devices.

To perform live IOP measurements, the posterior chamber was cannulated with a 25-gauge needle (Becton Dickinson, Franklin Lakes, N.J.) entering the pars plana to the center of the eye. The tip of the cannula was visually confirmed as beveled up before recording measurements. The cannula was connected to a DTXplus pressure transducer (Merit Medical, Singapore) and calibrated to a water height equivalent of 0 mmHg prior to insertion into the eye. The transducer signal was converted and recorded using a custom DAC converter hardware and software created by the Mayo Clinic Division of Engineering. (See, e.g., Roy Chowdhury U, Holman B H, Fautsch M P. A novel rat model to study the role of intracranial pressure modulation on optic neuropathies. PloS One 2013; 8: e82151.) The measurements were calibrated using a water column at various heights prior to use in the eye.

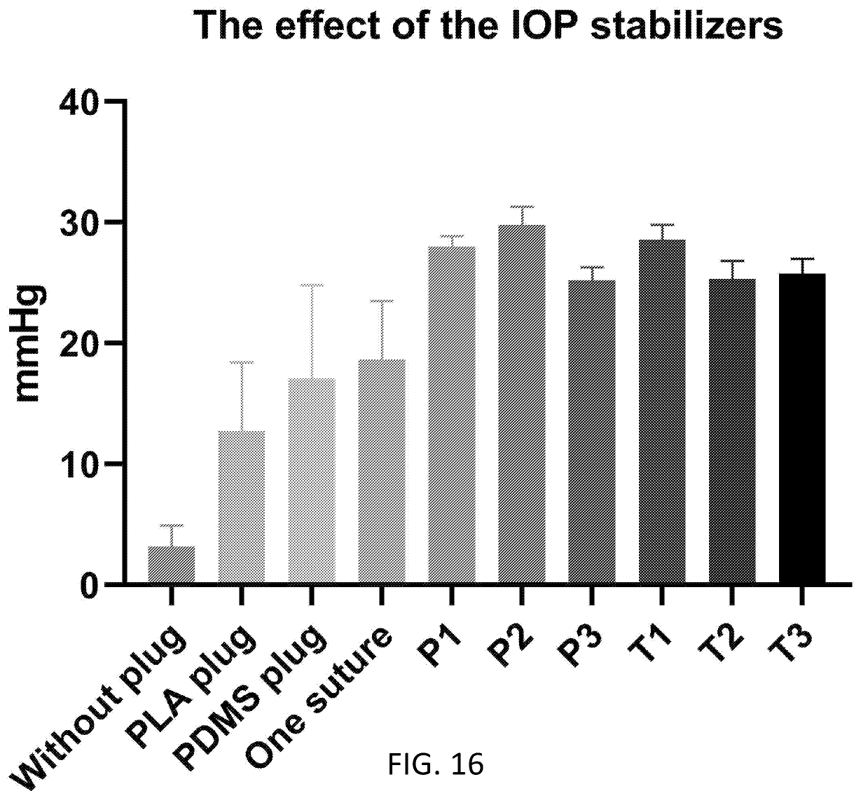

IOP measurements were recorded at key events during the procedure: background IOP prior to sclerotomy, the 3.6 mm scleral incision, insertion of the initial device tip, full insertion of the device to line up with the retinotomy, and removal of the insertion device. This procedure was repeated with an example implantation device of the disclosure without an IOP stabilizer (e.g., an example implantation device having tip assembly 150), with the PLA plug (e.g., first plug 180) and PDMS plug (e.g., second plug 186). The procedure was also repeated with various prototypes of the third implantation device 200. A final IOP measurement was taken after a single 8-0 Vicryl suture was placed in the middle of the scleral incision. IOP values shown in FIG. 16 were calculated from a consecutive ten seconds recording during each phase and averaged from n=10. Compliance of the setup, including the pig eye, was assumed to fall within the standard deviation of each group.

While this specification contains many specific implementation details, these should not be construed as limitations on the scope of any invention or of what may be claimed, but rather as descriptions of features that may be specific to particular embodiments of particular inventions. Certain features that are described in this specification in the context of separate embodiments can also be implemented in combination in a single embodiment. Conversely, various features that are described in the context of a single embodiment can also be implemented in multiple embodiments separately or in any suitable subcombination. Moreover, although features may be described herein as acting in certain combinations and even initially claimed as such, one or more features from a claimed combination can in some cases be excised from the combination, and the claimed combination may be directed to a subcombination or variation of a subcombination.

Similarly, while operations are depicted in the drawings in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system modules and components in the embodiments described herein should not be understood as requiring such separation in all embodiments, and it should be understood that the described program components and systems can generally be integrated together in a single product or packaged into multiple products.

Particular embodiments of the subject matter have been described. Other embodiments are within the scope of the following claims. For example, the actions recited in the claims can be performed in a different order and still achieve desirable results. As one example, the process depicted in the accompanying figures do not necessarily require the particular order shown, or sequential order, to achieve desirable results. In certain implementations, multitasking and parallel processing may be advantageous.

### OTHER EMBODIMENTS

In one aspect, this disclosure is directed to a device from implanting an implant. The device can include a handle assembly that includes an actuator, and a tip assembly located at a distal end of the handle assembly, where the tip assembly houses the implant. The actuator is configured to eject the implant from the tip assembly. In some cases, the actuator is a pneumatic actuator. In some cases, the pneumatic actuator is compatible with a vitrectomy unit. In some cases, the handle assembly receives the tip assembly. In some cases, the tip assembly is removably coupled to the distal end of the handle assembly. In some cases, the tip assembly is removably coupled to the distal end of the handle assembly via threads. In some cases, the tip assembly is disposable. In some cases, the tip assembly is preloaded with the implant. In some cases, the tip assembly includes a tip hub that couples to the handle assembly. In some cases, the tip hub includes a spring loaded pin. In some cases, the spring loaded pin is in communication with the actuator, and the spring loaded pin is configured to eject the implant from the tip assembly upon actuation of the spring loaded pin. In some cases, the tip assembly includes a tip housing and a tube extending between the tip hub and the tip housing. In some cases, the tube houses a guidewire extending between the tip hub and the tip housing. In some cases, the tip housing includes a plunger coupled to the guidewire, where plunger is in communication with the actuator, and the plunger is configured to eject the implant from the tip housing upon actuation of the plunger. In some cases, the tip housing is clear. In some cases, the tube is stainless steel. In some cases, the tube defines a radius of curvature. In some cases, the radius of curvature is about 3 mm to about 60 mm. In some cases, the tube includes a seal, and the seal is configured to inhibit eye fluid from entering the tip hub. In some cases, the implant is a RPE/fibrin hydrogel implant.

In another aspect, this disclosure is directed to a tip assembly for implanting an implant. The tip assembly includes a hub including an actuator, a tip housing configured to house the implant, and a tube extending between the hub and the tip housing. The actuator is configured to eject the implant from the tip housing. In some cases, the actuator is a spring loaded pin. In some cases, the spring loaded pin is in communication with a pneumatic actuator and the spring loaded pin is configured to eject the implant from the tip housing upon actuation of the spring loaded pin. In some cases, the tube houses a guidewire extending between the actuator of the hub and the tip housing. In some cases, the tip housing includes a plunger coupled to the guidewire. In some cases, the plunger is in communication with the actuator and the plunger is configured to eject the implant from the tip housing upon actuation of the plunger. In some cases, the tip housing is clear. In some cases, the tube is stainless steel. In some cases, the tube defines a radius of curvature. In some cases, the radius of curvature is about 3 mm to about 60 mm. In some cases, the tube includes a seal that inhibits eye fluid from entering the tip hub. In some cases, the actuator is a pneumatic actuator. In some cases, the pneumatic actuator is compatible with a vitrectomy unit. In some cases, the tip hub is removably coupled to a handle assembly. In some cases, the tip hub includes threads to removably couple the tip hub to the handle assembly. In some cases, the tip assembly is disposable. In some cases, the tip assembly is preloaded with the implant. In some cases, the implant is a RPE/fibrin hydrogel implant.

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1.-54. (canceled)

55. A device for implanting an implant, the device comprising:
a handle assembly comprising an actuator; and
a tip assembly at a distal end of the handle assembly, the tip assembly configured to house the implant,
wherein the actuator is configured to eject the implant from the tip assembly.

56. The device of claim 55, wherein the actuator is a pneumatic actuator that is compatible with a vitrectomy unit.

57. The device of claim 55, wherein the tip assembly comprises a tip hub configured to couple to the handle assembly.

58. The device of claim 57, wherein the tip hub comprises a spring-loaded pin.

59. The device of claim 58, wherein the spring-loaded pin is in communication with the actuator, wherein the spring-loaded pin is configured to eject the implant from the tip assembly upon actuation of the spring-loaded pin.

60. The device of claim 57, wherein the tip assembly further comprises a tip housing and a tube extending between the tip hub and the tip housing.

61. The device of claim 60, wherein the tube houses a guidewire extending between the tip hub and the tip housing.

62. The device of claim 61, wherein the tip housing comprises a plunger coupled to the guidewire, wherein the plunger is in communication with the actuator, wherein the plunger is configured to eject the implant from the tip housing upon actuation of the plunger.

63. The device of claim 60, wherein the tube houses a push rod extending between the hub and the tip housing.

64. The device of claim 63, wherein the push rod has a generally flattened, oval-shaped cross section.

65. The device of claim 60, wherein the tip housing is clear.

66. The device of claim 60, wherein the tube defines a radius of curvature.

67. The device of claim 66, wherein the radius of curvature is about 3 mm to about 60 mm.

68. The device of claim 60, wherein the tube further comprises a seal, wherein the seal is configured to inhibit eye fluid from entering the tip hub.

69. The device of claim 55, wherein the implant is a drug delivery implant, a gene therapy delivery implant, or a cell-based implant.

70. The device of claim 55, wherein the implant is a biological tissue.

71. The device of claim 55, wherein the implant comprises a matrix or a scaffold comprising a natural material, a synthetic material, or a hybrid material.

72. The device of claim 55, wherein the implant is a RPE/fibrin hydrogel implant.

73. The device of claim 55, further comprising a device holder, comprising:
a semi-open ring comprising a first radial arm and a second radial arm, the first and second radial arms extending from a back surface of the device holder;
a first side wall and a second side wall defining a guide channel, the first and second side walls extending from the back surface; and
a first holder arm and a second holder arm integrally connected to the back surface, the first and second holder arms defining an opening,
wherein the opening and the guide channel are configured to receive the tip assembly.

74. A tip assembly for implanting an implant, the tip assembly comprising:
a hub comprising an actuator;
a tip housing configured to house the implant; and
a tube extending between the hub and the tip housing,
wherein the actuator is configured to eject the implant from the tip housing.
